(19) United States
(12) Patent Application Publication
(10) Pub. No.: US 2007/0286882 A1
(43) Pub. Date: Dec. 13, 2007

(54) Solvent systems for coating medical devices

(76) Inventors: Yiwen Tang, San Jose, CA (US); Gina Zhang, Temecula, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/450,558

(22) Filed: Jun. 9, 2006

(51) Int. Cl. A61F 2/02; A61K 31/56; A61K 38/44; A61K 38/43; A61K 31/4745

(52) U.S. Cl. 424/423; 424/94.1; 424/94.4; 514/291; 514/171

(57) ABSTRACT

The present invention discloses a method of modulating drug release from a coating on a medical device, a medical device including a coating formed thereby, and a method of using the medical device for treating, preventing or ameliorating a medical condition.

### FIELD OF THE INVENTION

This invention is directed to the control of concentration gradients within polymeric matrices in the design of release profiles of agents from within these matrices.

### BACKGROUND

Biomaterials research is continuously striving to improve the compositions from which medical articles, such as medical devices and coatings for medical devices, are produced. An example of a medical article is an implantable medical device.

A stent is an example of an implantable medical device that can benefit from improvements such as, for example, a coating that can be used as a vehicle for delivering pharmaceutically active agents in a predictable manner. Stents can act as a mechanical intervention to physically hold open and, if desired, expand a passageway within a subject. Typically, a stent may be compressed, inserted into a small vessel through a catheter, and then expanded to a larger diameter once placed in a proper location. Examples of patents disclosing stents include U.S. Pat. Nos. 4,733,665, 4,800,882 and 4,886,062.

Stents play an important role in a variety of medical procedures such as, for example, percutaneous transluminal coronary angioplasty (PTCA), which is a procedure used to treat heart disease. In PTCA, a balloon catheter is inserted through a brachial or femoral artery, positioned across a coronary artery occlusion, inflated to compress atherosclerotic plaque and open the lumen of the coronary artery, deflated and withdrawn. Problems with PTCA include formation of intimal flaps or torn arterial linings, both of which can create another occlusion in the lumen of the coronary artery. Moreover, thrombosis and restenosis may occur several months after the procedure and create a need for additional angioplasty or a surgical by-pass operation. Stents are generally implanted to reduce occlusions, inhibit thrombosis and restenosis, and maintain patency within vascular lumens such as, for example, the lumen of a coronary artery.

Stents are also being developed to provide a local delivery of agents. Local delivery of agents is often preferred over systemic delivery of agents, particularly where high systemic doses are necessary to achieve an effect at a particular site within a subject—high systemic doses of agents can often create adverse effects within the subject. One proposed method of local delivery includes coating the surface of a medical article with a polymeric carrier and attaching an agent to, or blending it with, the polymeric carrier.

Agent-coated stents have demonstrated dramatic reductions in the rates of stent restenosis by inhibiting tissue growth associated with the restenosis. Restenosis, for example, is a very complicated process. Agents have been applied, alone and in combination, in an attempt to circumvent the process of restenosis. The process of restenosis in coronary artery disease is derived from a complex interplay of several implant-centered biological parameters. These are thought to be the combination of elastic recoil, vascular remodeling, and neo-intimal hyperplasia. Since restenosis is a multifactorial phenomenon, the local delivery of agents from a stent would benefit from the design of a release rate profile that would deliver agents as needed from the stent in a controlled and predictable manner. For example, one method of applying multiple agents involves blending the agents together in one formulation and applying the blend to the surface of a stent in a polymer matrix. A disadvantage of this method is that the agents are released from the matrix through a somewhat variable polymeric matrix morphology and compete with one another for release. As a result, delivery of the agents can be considered unpredictable.

Currently, compositions designed for use with existing methods of forming medical articles are often rejected because they produce polymeric matrices that are unable to meet particular performance characteristics. Often, the inability to meet particular performance characteristics results from combining components that are desirable independently but form undesirable morphologies that cannot meet the performance characteristics when formed into a polymeric matrix. Sometimes, the compositions produce polymeric matrices that are desirable but unpredictable in performance. Morphological changes are known to happen to medical articles during processing and storage, as well as after application in vivo. Unfortunately, the predictability of a medical article can rely on the ability to control these changes.

Liner polyesters of lactide and glycolide, for example, have been used for more than three decades for a variety of medical applications. Extensive research has been devoted to the use of these polymers as carriers for controlled drug delivery of a wide range of bioactive agents for human and animal use. For example, the have been used for the delivery of steroids, anticancer agents, peptides, proteins, antibiotics, anesthetics and vaccines. Investigations are undertaken to use poly(lactic acid) based materials as carriers for delivery of an agent such as everolimus from a drug delivery stent.

Controlling the performance of medical articles such as, for example, controlling the release of agents is an important aspect in the design of medical devices. In addition to providing a way to improve the bioactive, biobeneficial, and/or diagnostic results currently obtained from the administration of agents, control over the release rate of agents can assist in designing and maintaining the physical and mechanical properties of medical devices and coatings as well, and perhaps allow for the use of more desirable polymeric matrix components.

Accordingly, there is a need for control over the morphology of a polymeric matrix. The following embodiments address the above identified problems and needs.

### SUMMARY OF THE INVENTION

The present invention discloses a method of modulating drug release from a coating on a medical device, a medical device including a coating formed thereby, and a method of using the medical device for treating, preventing or ameliorating a medical condition. The method of modulating drug release includes:

(1) providing a composition comprising the polymer and the drug,

(2) dissolving the composition in solvent mixture that includes at least a first solvent and a second solvent to form a coating solution of the composition, where the boiling point of the first solvent and the boiling point of the second solvent are substantially different,

(3) applying the solution to a medical device, and

(4) forming a coating on the medical device.

The medical device can be, e.g., a stent. The polymer can be any biocompatible polymer such as poly(lactic acid) or a copolymer that comprises lactic acid. The drug can be any bioactive agent, for example, everolimus.

### BRIEF DESCRIPTION OF THE DRAWINGS

FIG. 1 shows everolimus release from a coating using acetone/ethanol (75/25) mixture as the coating solvent.

FIG. 2 shows everolimus release from a coating using methyl ethyl ketone/acetone (70/30) mixture as coating sovlent.

FIG. 3 shows scanning electron microscope (SEM) images of the coatings using acetone/ethanol (75/25) as the coating solvent.

FIG. 4 shows SEM images of the coatings coated using methyl ethyl keton/acetone (70/30) as the coating solvent.

FIGS. 5A and B shows SEM images of coatings coated using Dowanol (FIG. 5A) or Dowanol/acetone (60/40, FIG. 5B) as coating solvent.

FIGS. 6A-6F shows SEM images of coatings of configurations 1-6 having (1) a primer layer coated with tetrachloroethane (TCE)/acetone (80/20) as the coating solvent and (2) a drug layer coated using a solvent mixture that is TCE/acetone (40/60, FIG. 6A), TCE/acetone (60/40, FIG. 6B), TCE/acetone (80/20, FIG. 6C), Dowanol/dichloromethane (DCM) (30/70, FIG. 6D), Dowanol/DCM (50/50, FIG. 6E), and Dowanol/DCM (70/30, FIG. 6F).

### DETAILED DESCRIPTION OF THE INVENTION

Provided herein is a method of controlling morphology of a coating on a medical device (e.g., stent) to provide for controlled release of an agent, e.g., a drug, from the coating. The drug release rate can be controlled by controlling the microstructure of a coating. The microstructure of a coating can be varied and/or modified by selection of coating solvents.

The release rate of a drug from a coated film is related to the polymer/drug structure in the coated film, which, in turn, is related to the total solid content, conditions in forming the film, solvent used in the coating, and ratio of drug to polymer, etc. Under a given set of coating conditions, the nature of solvents plays an important role in forming the morphology of a coating.

As discussed in more detail below, the embodiments of the present invention generally encompass controlling the morphology of polymeric matrices in medical articles such as, for example, a medical device or a coating with the goal of controlling the performance characteristics of the matrices. The morphology of a polymeric matrix refers the way that the components of the matrix are arranged. More particularly, the present invention provides a method of controlling the release of an agent from a medical article and includes selecting a release rate for an agent, preparing a composition comprising a polymer and the agent in a solvent blend or combination, the solvent having different boiling points, solubility parameters, etc., and coating the composition on a medical device such as a drug delivery stent.

The control over the release of agents provides for control over, inter alia, the therapeutic, prophylactic, diagnostic, and ameliorative effects that are realized by a patient in need of such treatment. In addition, the control of the release rate of agents also has an effect upon the mechanical integrity of the polymeric matrix, as well as a relationship to a subject's absorption rate of the absorbable polymers. The polymeric matrices of the present invention can be used to form a medical article. A “medical article” can include, but is not limited to, a medical device or a coating for a medical device.

An “agent” can be a moiety that may be bioactive, biobeneficial, diagnostic, plasticizing, or have a combination of these characteristics. A “moiety” can be a functional group composed of at least 1 atom, a bonded residue in a macromolecule, an individual unit in a copolymer or an entire polymeric block. It is to be appreciated that any medical devices that can be improved through the teachings described herein are within the scope of the present invention.

The compositions and methods of the present invention apply to the formation of medical devices and coatings. Examples of medical devices include, but are not limited to, stents, stent-grafts, vascular grafts, artificial heart valves, foramen ovale closure devices, cerebrospinal fluid shunts, pacemaker electrodes, guidewires, ventricular assist devices, cardiopulmonary bypass circuits, blood oxygenators, coronary shunts (AXIUS™, Guidant Corp.), vena cava filters, and endocardial leads (FINELINE® and ENDOTAK®, Guidant Corp.). In some embodiments, the stents include, but are not limited to, tubular stents, self-expanding stents, coil stents, ring stents, multi-design stents, and the like. In other embodiments, the stents are metallic; low-ferromagnetic; non-ferromagnetic; biostable polymeric; biodegradable polymeric or biodegradable metallic. In some embodiments, the stents include, but are not limited to, vascular stents, renal stents, biliary stents, pulmonary stents and gastrointestinal stents.

### Control of Coating Morphology by Solvent Selection

In one aspect of the present invention, the morphology of the coating matrix containing a polymer (e.g., a PLA polymer), can be controlled by selection of a combination of solvents for forming the coating on a device (e.g., a stent). Selection of solvents can affect the release rate of a drug via, e.g., the following mechanism:

(1) evolution of a drug-polymer microstructural size and shape. This depends on drying rate, Volatility of solvent, humidity and hygroscopicity of the drug-polymer-solvent ternary system, and phase state of drug-polymer-solvent ternary system.

(2) evolution of a gradient of drug solid phase initial concentration. This depends on drying rate, volatility of solvent, humidity and hygroscopicity of the drug-polymer-solvent ternary system, and phase state of drug-polymer-solvent ternary system.

(3) The plasticization effect of the residual solvent altering both the mechanical property and diffusive property of the drug.

The coating (or casting) solvent used to form medical articles may be chosen based on several criteria including, for example, its polarity, ability to hydrogen bond, molecular size, volatility, biocompatibility, reactivity and purity. Other physical characteristics of the casting solvent may also be taken into account including the solubility limit of the polymer in the casting solvent; the presence of oxygen and other gases in the casting solvent; the viscosity and vapor pressure of the combined casting solvent and polymer; the ability of the casting solvent to diffuse through adjacent materials, such as an underlying material; and the thermal stability of the casting solvent.

One of skill in the art has access to scientific literature and data regarding the solubility of a wide variety of polymers. Furthermore, one of skill in the art will appreciate that the choice of casting solvent may begin empirically by calculating the Gibb's free energy of dissolution using available thermodynamic data. Such calculations allow for a preliminary selection of potential solvents to test in a laboratory. It is recognized that process conditions can affect the chemical structure of the underlying materials and, thus, affect their solubility in a casting solvent. It is also recognized that the kinetics of dissolution are a factor to consider when selecting a casting solvent, because a slow dissolution of an underlying material, for example, may not affect the performance characteristics of a product where the product is produced relatively quickly.

In some embodiments, the coating solvent is a combination of solvents. Generally, the solvents forming the combination have a substantially difference in boiling point. Solvents with a high boiling point evaporate slowly in the coating and/or casting process so that the coating formed with these coating solvents has a relatively fine and dense microstructure. Drug release rate from a coating thus formed is therefore relatively low. Conversely, solvents with a low boiling point evaporates fast in the coating or casting process so that the coating formed with these fast evaporating solvents has a relatively coarse microstructure. Drug release rate from a coating thus formed is therefore relatively high. Therefore, the drug release rate can be tuned and/or modified by selection of a combination of solvent(s) with a relatively high boiling point and solvent(s) with a relatively low boiling point. Therefore, a desired drug release rate can be obtained by varying the ratio of solvents with different boiling points.

In some embodiments, the solvents chosen to form a coating have a boiling point ranging from about 70° C. to about 90° C.

Exemplary casting solvents for use in the present invention include, but are not limited to, dimethyl acetamide (DMAC), dimethyl formamide (DMF), tetrahydrofuran (THF), TCE (1,1,2,2-tetrachloroethane), acetone, Dowanol™ (2-(2-ethoxyethoxy)ethanol), DCM (dichloromethane), MEK (methyl ethyl ketone), chloroform, ethanol, butanol, isopropyl acetate, pentane. Some other solvents that can be used include, but are not limited to, cyclohexanone, xylene, toluene, propylene glycol monomethyl ether, methyl butyl ketone, ethyl acetate, n-butyl acetate, and dioxane. Solvent mixtures can be used as well. Representative examples of the mixtures include, but are not limited to, DMAC and methanol (50:50 w/w); water, i-propanol, and DMAC (10:3:87 w/w); i-propanol and DMAC (80:20, 50:50, or 20:80 w/w); acetone and cyclohexanone (80:20, 50:50, or 20:80 w/w); acetone and xylene (50:50 w/w); acetone, xylene and FLUX REMOVER AMS® (93.7% 3,3-dichloro-1,1,1,2,2-pentafluoropropane and 1,3-dichloro-1,1,2,2,3-pentafluoropropane, and the balance is methanol with trace amounts of nitromethane; Tech Spray, Inc.) (10:40:50 w/w); and TCE and chloroform (80:20 w/w).

### Coating Compositions

The method described herein can be used to form any coating on a medical device (e.g., a stent), with or without a bioactive agent. The coating composition can include a biocompatible polymer(s), optionally a biobeneficial material, and/or a bioactive agent. The coating can be in any form of construct. For example, in some embodiments, the coating can have a drug reservoir, optionally with a topcoat and/or a primer layer and/or a finishing layer.

The biocompatible polymer useful in the present invention can be biodegradable or nondegradable and can be hydrophobic or hydrophilic. Representative examples of polymers that can be used to coat an implantable device in accordance with the present invention include, but are not limited to, poly(ester amide), ethylene vinyl alcohol copolymer (commonly known by the generic name EVOH or by the trade name EVAL), poly(hydroxyvalerate), poly(L-lactic acid), poly(L-lactide), poly(D,L-lactide), poly(L-lactide-co-D,L-lactide), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(D,L-lactide-co-glycolide) (PDLLAGA), poly(glycolic acid-co-trimethylene carbonate), polyphosphoester, polyphosphoester urethane, poly(amino acids), polycyanoacrylates, poly(trimethylene carbonate), poly(iminocarbonate), poly(butylene terephthalate-co-poly((ethylene glycol) (PEG)-terephthalate), polyurethanes, polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides such as vinylidene fluoride based homo or copolymer under the trade name Solef™ or Kynar™, for example, polyvinylidene fluoride (PVDF) or poly(vinylidene-co-hexafluoropropylene) (PVDF-co-HFP) and polyvinylidene chloride, polyacrylonitrile, polyvinyl ketones, polyvinyl aromatics such as polystyrene, polyvinyl esters, such as polyvinyl acetate, copolymers of vinyl monomers with each other and olefins such as ethylene-methyl methacrylate copolymers, acrylonitrile-styrene copolymers, ABS resins, and ethylene-vinyl acetate copolymers, polyamides such as Nylon 66 and polycaprolactam, alkyd resins, polycarbonates, polyoxymethylenes, polyimides, polyethers, poly(glyceryl sebacate), poly(propylene fumarate), epoxy resins, polyurethanes, rayon, rayon-triacetate, cellulose acetate, cellulose butyrate, cellulose acetate butyrate, cellophane, cellulose nitrate, cellulose propionate, cellulose ethers, and carboxymethyl cellulose.

A preferred biocompatible, hydrophobic polymer is a polyester, such as one of poly(D,L-lactic acid) (PDLLA), poly(L-lactic acid) (PLLA), poly(D-lactic acid) (PDLA), poly(D,L-lactic acid-co-glycolic acid) (PDLLGA), poly(glycolic acid) (PGA), polyhydroxyalkanoates (PHA), poly(3-hydroxybutyrate) (PHB), poly(3-hydroxybutyrate-co-3-hydroxyvalerate), poly((3-hydroxyvalerate), poly(3-hydroxyhexanoate), poly(4-hydroxybutyrate), poly(4-hydroxyvalerate), poly(4-hydroxyhexanoate), polycaprolactone (PCL), poly(ester amide), poly(ethylene-co-vinyl alcohol) (EVAL), PVDF, copolymers such as PVDF-HFP, PEG-PLA, PCL-PLA where the monomer lactic acid can be either a D- or L-stereo isomer, a racemic mixture, or a blend of the D- and L-isomer, poly(urethanes), or a combination thereof.

The biobeneficial material that can be used in the present invention can be a polymeric material or non-polymeric material. The biobeneficial material is preferably flexible and biocompatible and/or biodegradable (a term which includes bioerodable, biodegradable and bioabsorbable), more preferably non-toxic, non-antigenic and non-immunogenic. A biobeneficial material is one which enhances the biocompatibility of a device by being non-fouling, hemocompatible, actively non-thrombogenic, or anti-inflammatory, all without depending on the release of a pharmaceutically active agent.

Representative biobeneficial materials include, but are not limited to, polyethers such as poly(ethylene glycol), copoly(ether-esters) (e.g. PEO/PLA); polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as hydroxyethyl methacrylate (HEMA), hydroxypropyl methacrylate (HPMA), hydroxypropylmethacrylamide, poly (ethylene glycol) acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as fibrin, fibrinogen, cellulose, starch, collagen, dextran, dextrin, hyaluronic acid, fragments and derivatives of hyaluronic acid, heparin, fragments and derivatives of heparin, glycosamino glycan (GAG), GAG derivatives, polysaccharide, elastin, chitosan, alginate, silicones, and a combination thereof. In some embodiments, the polymer can exclude any one of the aforementioned polymers.

In a preferred embodiment, the biobeneficial material is a block copolymer having flexible poly(ethylene glycol) and poly(butylene terephthalate) blocks (PEGT/PBT) (e.g., PolyActive™). PolyActive™ is intended to include AB, ABA, BAB copolymers having such segments of PEG and PBT (e.g., poly(ethylene glycol)-block-poly(butyleneterephthalate)-block poly(ethylene glycol) (PEG-PBT-PEG).

Representative hydrophilic materials that can be used include hyaluronate, heparin, polyethylene glycol, polyalkene oxides, block copolymer poly(ethylene glycol terephtalate)/poly(butylenes terephtalate) (PEGT/PBT) (PolyActive™), phosphoryl choline, poly(aspirin), poly (N-vinylpyrrolidone) (PNVP), SIS-PEG, polystyrene-PEG, polyisobutylene-PEG, PCL-PEG, PLA-PEG, PMMA-PEG, PDMS-PEG, PVDF-PEG, SIS-hyaluronic acid (HA), polystyrene-HA, polyisobutylene-HA, PCL-HA, PLA-HA, PMMA-HA, PVDF-HA, SIS-heparin, polystyrene-heparin, polyisobutylene-heparin, PCL-heparin, PLA-heparin, PMMA-heparin, PVDF-heparin, and a combination thereof.

Bioactive agents that can be used in the present invention can be any agent which is a therapeutic, prophylactic, or diagnostic agent. These agents can have anti-proliferative or anti-inflammmatory properties or can have other properties such as antineoplastic, antiplatelet, anti-coagulant, anti-fibrin, antithrombonic, antimitotic, antibiotic, antiallergic, antioxidant as well as cystostatic agents. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of anti-proliferative agents include rapamycin and its functional or structural derivatives, 40-O-(2-hydroxy)ethyl-rapamycin (everolimus), and its functional or structural derivatives, paclitaxel and its functional and structural derivatives. Examples of rapamycin derivatives include methyl rapamycin (ABT-578), 40-O-(3-hydroxy)propyl-rapamycin, 40-O-[2-(2-hydroxy)ethoxy]ethyl-rapamycin, and 40-O-tetrazole-rapamycin. Examples of paclitaxel derivatives include docetaxel. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax ä (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of anti-inflammatory agents including steroidal and non-steroidal anti-inflammatory agents include tacrolimus, dexamethasone, clobetasol, and a combination thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include alpha-interferon, pimecrolimus, imatinib mesylate, midostaurin, bioactive RGD, and genetically engineered epithelial cells. The foregoing substances can also be used in the form of prodrugs or co-drugs thereof. The foregoing substances are listed by way of example and are not meant to be limiting. Other active agents which are currently available or that may be developed in the future are equally applicable.

The dosage or concentration of the agent required to produce a favorable therapeutic effect should be less than the level at which the agent produces toxic effects and greater than the level at which non-therapeutic results are obtained. The dosage or concentration of the agent required can depend upon factors such as the particular circumstances of the patient, the nature of the tissues being delivered to, the nature of the therapy desired, the time over which the ingredient administered resides at the vascular site, and if other agents are employed, the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

### Examples of Implantable Device

As used herein, an implantable device may be any suitable medical substrate that can be implanted in a human or veterinary patient. Examples of such implantable devices include self-expandable stents, balloon-expandable stents, stent-grafts, grafts (e.g., aortic grafts), artificial heart valves, cerebrospinal fluid shunts, pacemaker electrodes, endocardial leads (e.g., FINELINE and ENDOTAK, available from Guidant Corporation, Santa Clara, Calif.), and implantable pump. The underlying structure of the device can be of virtually any design. The device can be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or a combination thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers could also be used with the embodiments of the present invention. In some embodiments, a bioabsorbable or bioerodable stent is used to carry HDL, recombinantn HDL or HDLm.

### Method of Use

In accordance with embodiments of the invention, a coating formed of the various described embodiments can be formed on an implantable device or prosthesis, e.g., a stent. For coatings including one or more active agents, the agent will retain on the medical device such as a stent during delivery and expansion of the device, and released at a desired rate and for a predetermined duration of time at the site of implantation. Preferably, the medical device is a stent. A stent having the above-described coating is useful for a variety of medical procedures, including, by way of example, treatment of obstructions caused by tumors in bile ducts, esophagus, trachea/bronchi and other biological passageways. A stent having the above-described coating is particularly useful for treating occluded regions of blood vessels caused by atherosclerosis, abnormal or inappropriate migration and proliferation of smooth muscle cells, thrombosis, and restenosis. Stents may be placed in a wide array of blood vessels, both arteries and veins. Representative examples of sites include the iliac, renal, and coronary arteries.

For implantation of a stent, an angiogram is first performed to determine the appropriate positioning for stent therapy. An angiogram is typically accomplished by injecting a radiopaque contrasting agent through a catheter inserted into an artery or vein as an x-ray is taken. A guidewire is then advanced through the lesion or proposed site of treatment. Over the guidewire is passed a delivery catheter which allows a stent in its collapsed configuration to be inserted into the passageway. The delivery catheter is inserted either percutaneously or by surgery into the femoral artery, brachial artery, femoral vein, or brachial vein, and advanced into the appropriate blood vessel by steering the catheter through the vascular system under fluoroscopic guidance. A stent having the above-described coating may then be expanded at the desired area of treatment. A post-insertion angiogram may also be utilized to confirm appropriate positioning.

### EXAMPLES

The following examples are provided to further teach the concepts and embodiments of the present invention.

### Example 1

### Coating with Acetone/Ethanol Solvent Mixture

### Materials and Methods

### Coating Compositions

Acetone/ETOH (75/25)—3 lots were made
DL-PLA/everolimus ratio: 1:1
Solvent: Acetone/EtOH: 75/25;
Total solid percent: 4%
Stent platform: Vision 18 mm small
Baking condition: 60° C. for 2 hours

Acetone/MEK (30/70)—3 lots were made
DL-PLA/everolimus ratio: 1:1
Solvent: Acetone/MEK: 30/70
Total solid percent: 4%
Stent platform: Vision 18 mm small
Baking condition: 60° C. for 2 hours

The stents were coated, baked, and then tested at a terminal weight stage. The stents were then tested according to the procedures below.

### Methods:

Dry expansion to RBP followed by the SEM (n=3 for each lot). Total content was measured in XL-80N, n=12 for each lot. Results for stents coated using acetone/ethanol solvent mixture are shown in Table 1.

TABLE 1
Total contents of coatings using acetone/ethanol solvent mixture
(75/25) as the coating solvent.
40727E1 Group-1

| | Sample# | | | | | | | | |
|---|---|---|---|---|---|---|---|---|---|
| | 1 | 2 | 3 | 4 | 5 | 6 | Average | SD | RSD |
| HPLC Recovered(ug) | 431.83 | 422.43 | 425.22 | 428.71 | 425.27 | 428.65 | 427.02 | 3.35 | 1% |
| Coating Weight(ug) | 889.00 | 879.00 | 883.00 | 883.00 | 878.00 | 887.00 | 883.17 | 4.31 | 0% |
| Theoretical(ug/stent) | 444.50 | 439.50 | 441.50 | 441.50 | 439.00 | 443.50 | 441.58 | 2.15 | 0% |
| % Recovered | 97.1% | 96.1% | 96.3% | 97.1% | 96.9% | 96.7% | 0.97 | 0.00 | 0% |

40727E2 Group-2

| | Sample# | | | | | | | | |
|---|---|---|---|---|---|---|---|---|---|
| | 1 | 2 | 4 | 7 | 8 | 10 | Average | SD | RSD |
| HPLC Recovered(ug) | 422.74 | 433.55 | 539.69 | 429.07 | 530.83 | 429.85 | 464.29 | 55.16 | 12% |
| Coating Weight(ug) | 880.00 | 910.00 | 896.00 | 906.00 | 883.00 | 909.00 | 897.33 | 13.26 | 1% |
| Theoretical(ug/stent) | 440.00 | 455.00 | 448.00 | 453.00 | 441.50 | 454.50 | 448.67 | 6.63 | 1% |
| % Recovered | 96.1% | 95.3% | 120.5% | 94.7% | 120.2% | 94.6% | 1.04 | 0.13 | 13% |

40727E3 Group-3

| | Sample# | | | | | | | | |
|---|---|---|---|---|---|---|---|---|---|
| | 4 | 5 | 6 | 7 | 8 | 9 | Average | SD | RSD |
| HPLC Recovered(ug) | 431.33 | 419.60 | 415.84 | 422.98 | 426.34 | 426.38 | 423.75 | 5.50 | 1% |
| Coating Weight(ug) | 879.00 | 871.00 | 889.00 | 899.00 | 900.00 | 898.00 | 889.33 | 12.04 | 1% |
| Theoretical(ug/stent) | 439.50 | 435.50 | 444.50 | 449.50 | 450.00 | 449.00 | 444.67 | 6.02 | 1% |
| % Recovered | 98.1% | 96.3% | 93.6% | 94.1% | 94.7% | 95.0% | 0.95 | 0.02 | 2% |

### The Total content is above 94%.

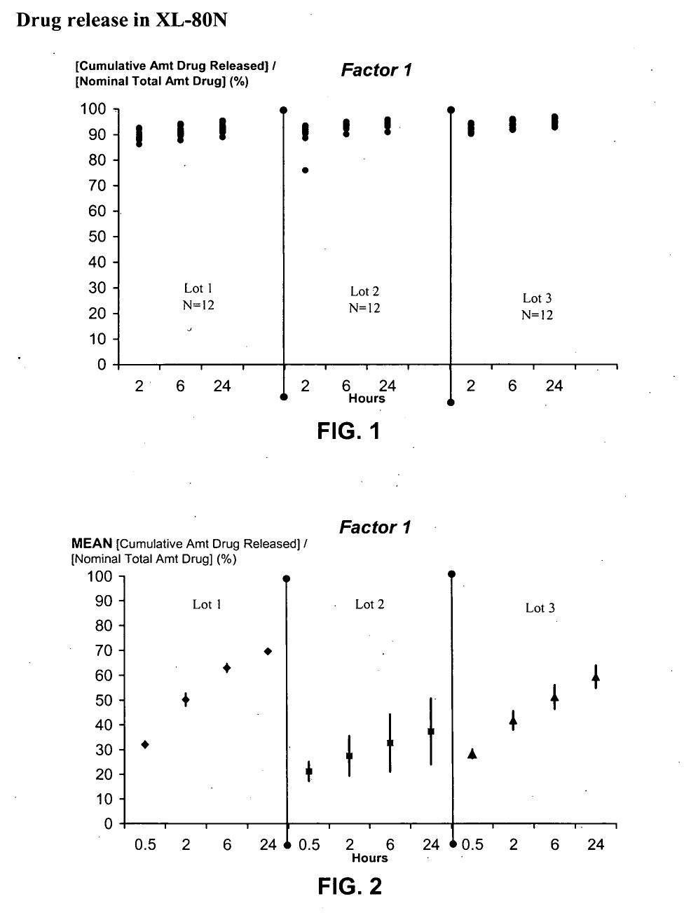

Drug release from the stents was tested in XL-80N. The results are shown were shown in FIG. 1.

Total contents for coatings coated using methyl ethyl ketone/acetone (70/30) mixture as coating solvent are shown in Table 2.

TABLE 2
40728E1 Group-1

| | Sample# | | | | | | | | |
|---|---|---|---|---|---|---|---|---|---|
| | 1 | 2 | 4 | 5 | 7 | 8 | Average | SD | RSD |
| HPLC Recovered(ug) | 418.28 | 415.61 | 407.70 | 410.90 | 416.05 | 414.07 | 413.77 | 3.85 | 1% |
| Coating Weight(ug) | 896.00 | 885.00 | 874.00 | 899.00 | 893.00 | 894.00 | 890.17 | 9.20 | 1% |
| Theoretical(ug/stent) | 448.00 | 442.50 | 437.00 | 449.50 | 446.50 | 447.00 | 445.08 | 4.60 | 1% |
| % Recovered | 93.4% | 93.9% | 93.3% | 91.4% | 93.2% | 92.6% | 0.93 | 0.01 | 1% |

40728E2 Group-2

| | Sample# | | | | | | | | |
|---|---|---|---|---|---|---|---|---|---|
| | 2 | 3 | 4 | 5 | 6 | 7 | Average | SD | RSD |
| HPLC Recovered(ug) | 422.05 | 411.43 | 418.89 | 422.87 | 420.18 | 424.04 | 419.91 | 4.55 | 1% |
| Coating Weight(ug) | 889.00 | 870.00 | 876.00 | 898.00 | 887.00 | 895.00 | 885.83 | 10.87 | 1% |
| Theoretical(ug/stent) | 444.50 | 435.00 | 438.00 | 449.00 | 443.50 | 447.50 | 442.92 | 5.44 | 1% |
| % Recovered | 94.9% | 94.6% | 95.6% | 94.2% | 94.7% | 94.8% | 0.95 | 0.00 | 1% |

40728E3 Group-3

| | Sample# | | | | | | | | |
|---|---|---|---|---|---|---|---|---|---|
| | 1 | 2 | 3 | 4 | 6 | 8 | Average | SD | RSD |
| HPLC Recovered(ug) | 430.19 | 413.07 | 404.40 | 410.88 | 296.00 | 416.97 | 395.25 | 49.37 | 12% |
| Coating Weight(ug) | 911.00 | 874.00 | 857.00 | 868.00 | 936.00 | 887.00 | 888.83 | 29.62 | 3% |
| Theoretical(ug/stent) | 455.50 | 437.00 | 428.50 | 434.00 | 468.00 | 443.50 | 444.42 | 14.81 | 3% |
| % Recovered | 94.4% | 94.5% | 94.4% | 94.7% | 63.2% | 94.0% | 0.89 | 0.13 | 14% |

### The total contents for Group 1 and Group 2 coatings are above 91%. The total contents for Group 3 coatings are generally above 94% except for Sample No. 6, which has a total content of 63.2%.

Drug release in XL-80N from coatings coated using methyl ethyl ketone/acetone mixture (70/30) is shown in FIG. 2.

The total content results for both coating were normal.

The drug release profile for the coating with ACE/EtOH was fast for all the three lots, indicating a drug release without control. For these three lots, the standard deviation was also very small—basically because the drug was dumped out and therefore caused less release variation.

For the MEK/ACE system, the drug release profile showed to be in a controlled manner. The first time point was 0.5 hour and the drug release was under 35%. However, the release variation varied a lot between the lots, e.g., for lot 1, the standard deviation is very small, but the standard deviation became large in lot 2. We cannot conclude if this lot-to-lot variability is due to lack of control in the CER where they were processed, or if it is due to some inherent property of the formulation.

### Scanning Electron Microscope (SEM) Studies

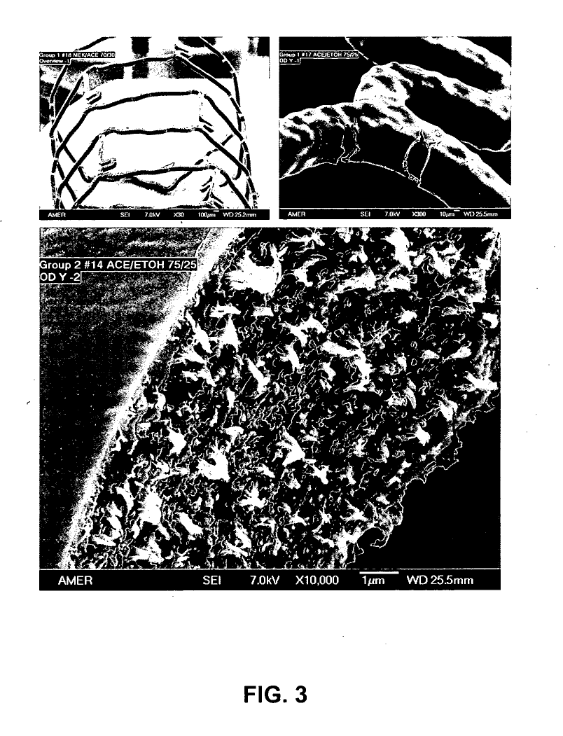

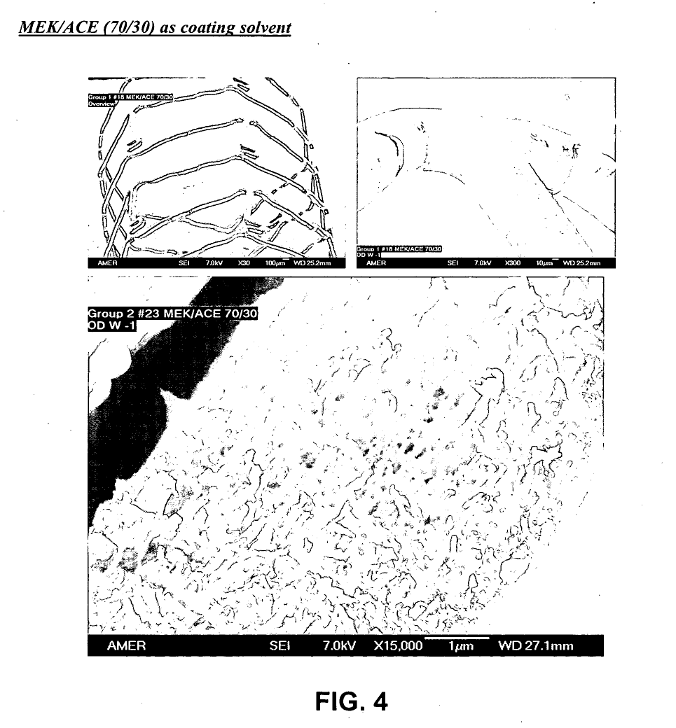

The coatings formed above were subjected to SEM study. FIG. 3 shows SEM the typical images of the coatings coated using acetone/ethanol (75/25) as the coating solvent. FIG. 4 shows the typical SEM images of the coatings coated using methyl ethyl keton/acetone (70/30) as the coating solvent. Both of the coating microstructure showed microphase separation, and, the SEM images of coatings coated using the two coating solvents look very similar.

### Discussions

Two formulations were spray coated onto Vision stent, using same coating parameters. From SEM images, both of them showed phase separation, although in a much more homogeneous pattern than those of hand coated, or auto coated stents.

The drug release profile for these two coating in XL-80N was significantly different. The coating with ACE/EtOH (75/25) had a fast release where the drug almost completely released at 2 hours. Although the standard deviations for this system were small for all the three lots, this is mostly due to the fact that the drug was released quickly. The drug release profile for the coating with MEK/ACE (70/30) showed more release rate controll. The first time point at 0.5 hour had a release smaller than 35%. At 24 hours, the drug release was about 70%. However, the standard deviations varied from lot to lot. For lot 1, the standard deviations were very small. However, the standard deviation for lot 2 was very large. This may suggest that there was manufacturing variability in the coating process.

From the auto coating formulation study, the drug release for the MEK/ACE (70/30) is summarized as below (Table 3):

TABLE 3

| 2 hr | 24 hr | 48 hr |
|---|---|---|
| Ave: 11%, | Ave: 26%, | Ave: 37%, |
| stdev: 4%, | stdev: 13%, | stdev: 17%, |
| RSD: 37% | RSD: 52% | RSD: 48% |

The spray coated stents in this study were tested without down stream processing, therefore corresponding to the terminal weight samples by formulation group. Comparing to their data, the spray coated stents had a much faster release. As for the release variation, spray coat lot 1 had smaller standard deviation than the auto coated stents.

The coating thickness in this spray coating was designed to be similar to the auto coating. If assuming the spray coating is evenly distributed onto the OD, ID and sidewall, the coating thickness is about 7.6 um. Usually the OD had thicker coating, and therefore the thickness on the OD could be about 10 um which is about the same as that for the auto coated stents.

The total surface area for Vision 18 mm small stent is 0.87 cm². Based on the SEM for the auto coated stents (MEK/ACE formulation), at least 80% of the side wall was covered by the coating, and therefore the total coated surface area is about 0.70 cm². As the total surface area are not that much difference, the difference of the drug release profile in between the spray coated and auto coated system can be attributed to factors such as the degree of phase separation, the chemical components in each phases for these two different systems, etc.

In addition to the above studied acetone/ethanol (75/25) and MEK/acetone (70/30) coating solvent systems, spray coated systems using pure acetone as the coating solvent were also studied (systems 1-4 using PLA/drug (D:P=1:1)), as shown below:
System 1. Acetone as the only solvent (4% solid) spray coated onto BVS stent (surface area=1.74 cm²), 300 μg was coated onto this kind of stent
System 2. Acetone as the only solvent (4% solid) spray coated onto Vision 12 mm small stent (surface area=0.56 cm²), 600 μg was coated onto this kind of stent
System 3. Acetone/Ethanol (75/25) as the solvents (4% solid) spray coated onto Vision 18 mm small stent (surface area=0.87 cm²), 900 μg was coated onto this kind of stent
System 4. MEK/acetone (70/30) as the solvent (4% solid) spray coated onto Vision 18 mm small stent (surface area=0.87 cm²), 900 μg was coated onto this kind of stent.

The drug release profile for these four systems in XL-80N has been very different, although their microstructure on the basis of SEM images looked similar. The drug release rate is as following: System 3>system 1>system 4>system 2.

### Example 2

### Study of Effect of Coating Solvent on Drug Release Rate

Drug release rate of everolimus from a PLA coating coated with different solvent systems was studied as described below.

Study 1. The Dowanol/acetone coating system. Table 4 summarizes the coating configurations in this study.

TABLE 4
Coating configurations in the study of solvent effects using
Dowanol/acetone coating system

| | Configuration 1 | Configuration 2 | Configuration 3 |
|---|---|---|---|
| Matrix | Solution 1 | Solution 2 | Solution 3 |
| Drug/PLA | Drug/PLA (1:1) in | Drug/PLA (1:1) | Drug/PLA (1:1) |
| (1:1) | 100% Dowanol | in Dowanol/ | in Dowanol/ |
| | 360 μg | acetone 80/20 | acetone 60/40 |
| | | 360 μg | 360 μg |
| No. of stents | 10 stents | 10 stents | 10 stents |

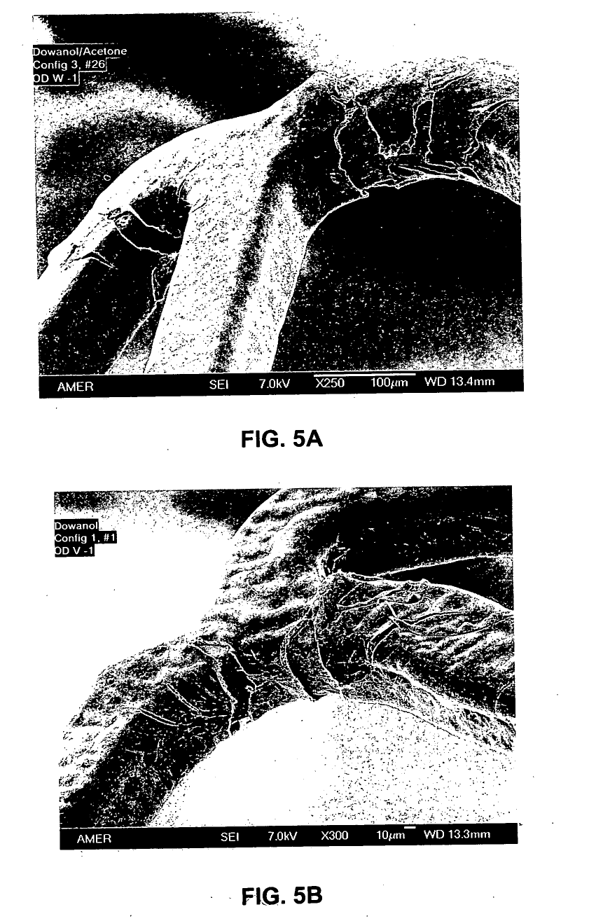

SEM images of coatings of configuration 1 and configuration 3 are shown in FIGS. 5A (Configuration 1) and 5B (Configuration 3).

Results at 24 hours:

Configuration 1: 92% (RSD=1%) was released;

Configuration 2: 92% (RSD=2%) was released;

Configuration 3: 93% (RSD=1%) was released;

Note: For spray coated stent with acetone as solvent, 8%±7% was released at 24 hours in post stenting (PS).

Study 2. The 1.1,2,2-tetrachloroethane (TCE), Dowanol, acetone, and dichloromethane (DCM) coating system. The coating configurations are summarized in Table 5.

TABLE 5
Coating configurations

| | | | # of |
|---|---|---|---|
| | Primer Coat | Matrix Coat | Unit |
| Configuration 1 | PLA in TCE/Acetone | Drug/PLA (1:1) in | 15 |
| | (80/20) | TCE/Acetone (40/60) | |
| | 80 ug | 370 ug | |
| | Solution 1 | Solution 2 | |
| Configuration 2 | PLA in TCE/Acetone | Drug/PLA (1:1) in | 15 |
| | (80/20) | TCE/Acetone (60/40) | |
| | 80 ug | 370 ug | |
| | Solution 1 | Solution 3 | |
| Configuration 3 | PLA in TCE/Acetone | Drug/PLA (1:1) in | 15 |
| | (80/20) | TCE/Acetone (80/20) | |
| | 80 ug | 370 ug | |
| | Solution 1 | Solution 4 | |
| Configuration 4 | PLA in TCE/Acetone | Drug/PLA (1:1) in | 15 |
| | (80/20) | Dowanol/DCM (30/70) | |
| | 80 ug | 370 ug | |
| | Solution 1 | Solution 5 | |
| Configuration 5 | PLA in TCE/Acetone | Drug/PLA (1:1) in | 15 |
| | (80/20) | Dowanol/DCM (50/50) | |
| | 80 ug | 370 ug | |
| | Solution 1 | Solution 6 | |
| Configuration 6 | PLA in TCE/Acetone | Drug/PLA (1:1) in | 15 |
| | (80/20) | Dowanol/DCM (70/30) | |
| | 80 ug | 370 ug | |
| | Solution 1 | Solution 7 | |

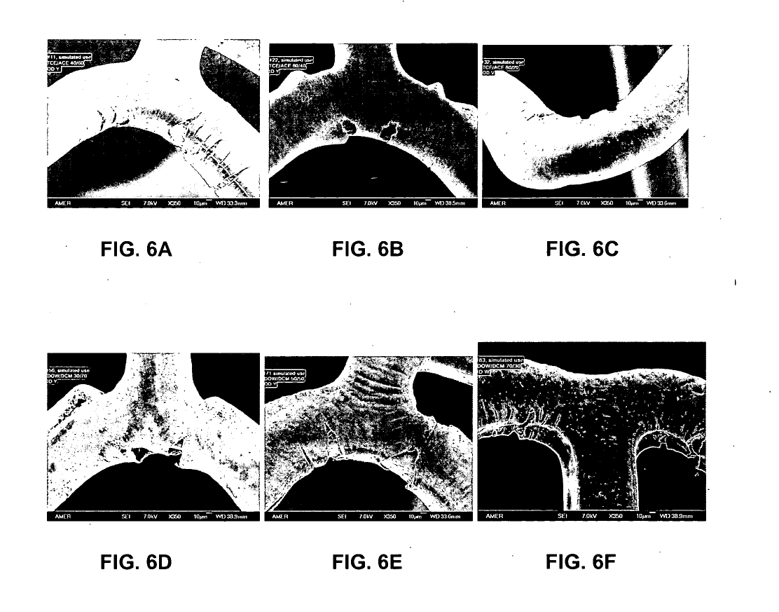

SEM images of coatings of configurations 1-6 are shown in FIGS. 6A-6F: FIG. 6A (Configuration 1), FIG. 6B (Configuration 2), FIG. 6C (Configuration 3), FIG. 6D (Configuration 4), FIG. 6E (Configuration 5), and FIG. 6F (Configuration 6).

Drug release results:

The drug release rate profiles of the stents coated according to the coating configurations in Table 5 were measured at 24 hours and 72 hours after implantation. The results are summarized below in Table 6. The release profiles of the stents by acetone/spray and Everest coating were measured as comparison.

TABLE 6

| Config. | 24 hours | 72 hours |
|---|---|---|
| 1 | 39.5% (RSD = 18.6%) | 47.6% (RSD = 13.2%) |
| 2 | 27.3% (RSD = 21.6%) | 31.5% (RSD = 6.2%) |
| 3 | 21.2% (RSD = 11.7%) | 23.2% (RSD = 5.8%) |
| 4 | 93.2% (RSD = 0.7%) | 93.4% (RSD = 1.3%) |
| 5 | 90.5% (RSD = 0.7%) | 90.5% (RSD = 1.5%) |
| 6 | 90.9% (RSD = 0.3%) | 90.5% (RSD = 0.5%) |
| Acetone/spray | 8% (RSD = 15%) | 10% (RSD = 16%) |
| Everest | 10% (RSD = 23%) | 15% (RSD = 30%) |

Studies 1 and 2 show that, for the spray coated PLA/everolimus coating, different solvent systems lead to different drug release rate.

While particular embodiments of the present invention have been shown and described, those skilled in the art will note that variations and modifications can be made to the present invention without departing from the spirit and scope of the teachings. A multitude of embodiments that include a variety of chemical compositions, polymers, agents and methods have been taught herein. One of skill in the art is to appreciate that such teachings are provided by way of example only and are not intended to limit the scope of the invention. The embodiments for the IM profiles that are taught herein are not meant to be limiting, since the IM profiles possible are virtually limitless in variety. The IM profiles taught in the present invention can be incorporated into any medical article.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

We claim:

1. A method for modulation of drug release from a coating comprising a polymer and a drug, comprising:
providing a composition comprising the polymer and the drug,
dissolving the composition in solvent mixture that includes at least a first solvent and a second solvent to form a coating solution of the composition, where the boiling point of the first solvent and the boiling point of the second solvent are substantially different,
applying the solution to a medical device, and
forming a coating on the medical device.

2. The method of claim 1, wherein the drug is selected from the group consisting of paclitaxel, docetaxel, estradiol, nitric oxide donors, super oxide dismutases, super oxide dismutases mimics, 4-amino-2,2,6,6-tetramethylpiperidine-1-oxyl (4-amino-TEMPO), tacrolimus, dexamethasone, rapamycin, rapamycin derivatives, 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), clobetasol, pimecrolimus, imatinib mesylate, midostaurin, prodrugs thereof, co-drugs thereof, and a combination thereof.

3. The method of claim 1, wherein the composition further comprises a biobeneficial material.

4. The method of claim 1, wherein the medical device is stent.

5. The method of claim 4, wherein the polymer is poly(lactic acid) (PLA) or a copolymer comprising lactic acid.

6. The method of claim 5, wherein the drug is 40-O-(2-hydroxy)ethyl-rapamycin (everolimus).

7. A medical device having a coating formed according to the method of claim 1.

8. A medical device having a coating formed according to the method of claim 2.

9. A medical device having a coating formed according to the method of claim 3.

10. A stent having a coating formed according to the method of claim 4.

11. A stent having a coating formed according to the method of claim 5.

12. A stent having a coating formed according to the method of claim 6.

13. A method for treating, preventing or ameliorating a medical condition, comprising implanting in a human being the medical device of claim 7,
wherein the medical condition is selected from the group consisting of atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof.

14. A method for treating, preventing or ameliorating a medical condition, comprising implanting in a human being the stent of claim 12,
wherein the medical condition is selected from the group consisting of atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof.
